(19) United States
(12) Patent Application Publication
(10) Pub. No.: US 2005/0191356 A1
(43) Pub. Date: Sep. 1, 2005

(54) Porous and non-porous matrices based on chitosan and hydroxy carboxylic acids

(76) Inventors: Andrea Pahmeier, Zossen (DE); Birger Hahnemann, Zossen (DE); Philipp Sperling, Berlin (DE); Thomas Lotzbeyer, Eching (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/678,751

(22) Filed: Oct. 6, 2003

Related U.S. Application Data
(63) Continuation of application No. PCT/EP02/03798, filed on Apr. 5, 2002.

(30) Foreign Application Priority Data
Apr. 6, 2001 (DE) 101 17 234.6

(51) Int. Cl. A61K 9/70; A61K 9/14

(52) U.S. Cl. 424/488; 424/443

(57) ABSTRACT

The invention relates to biocompatible matrices based on chitosan and hydroxy carboxylic acids, to multilayer systems comprising these matrices and to applications of such matrices.

The invention relates to biocompatible matrices based on chitosan and hydroxy carboxylic acids, to multilayer systems comprising these matrices and to applications of such matrices.

Considerable successes have been achieved in recent years in the area of medical transplants. However, problems arise through the small amounts of donor organs available and through rejection reactions caused by heterologous organs. A further problem is that pathogens can also be transmitted with heterologous donor organs. Attempts have therefore been made to culture artificial organs from cell cultures on a three-dimensional matrix which can be shaped according to requirements, for example as an ear. This artificial organ or body part can then be transplanted and, if endogenous cells are used, no rejection reaction occurs.

Chitosan has attracted increasing interest as a promising matrix material. Chitosan is a partly deacetylated chitin and is obtained from exoskeletons of arthropods. It is an aminopolysaccharide (poly-1-4-glucosamine) which is used for example in the medical sector as suture material or for encapsulating drugs. Its advantage is that it can be completely absorbed by the body. Chitosan can be dissolved in water in the slightly acid range (pH<6) through protonation of the free amino groups. In the alkaline range (pH>7) it precipitates again from the aqueous solution. Chitosan can be purified and processed under mild conditions through this pH-dependent mechanism.

U.S. Pat. No. 5,871,985proposes a vehicle for transplantation into a patient which consists of a matrix into which cells have grown. This is done by firstly preparing a solution of chitosan comprising living cells. This solution is then enclosed in a semipermeable membrane in order to form the carrier. The chitosan is precipitated and forms an uncrosslinked matrix in which the cells are dispersed.

Madihally et al. (Biomaterials 1999; 20(12), pages 1133-1142) describes a matrix for tissue generation. Chitosan which is 85-90% deacetylated is for this purpose dissolved in 0.2 M acetic acid to give solutions having a chitosan content of from 1 to 3% by weight. The solution is frozen and the water and the excess acetic acid are removed by lyophilization.

German patent application 199 48 120.2 discloses a method for producing a biocompatible three-dimensional matrix, where an aqueous solution of a chitosan and of an acid, in particular a hydroxy carboxylic acid, which is present in excess is frozen, and the water is removed by sublimation under reduced pressure, with the excess acid being removed, in particular neutralized, before the freezing or after the removal of the water by sublimation. In addition, a matrix which can be obtained by the method and which can be used for producing implants is disclosed.

Based on this knowledge, it was the object of the present invention to provide novel matrix forms or/and applications of a matrix based on chitosan and an acid, in particular a hydroxy carboxylic acid.

A first aspect of the present invention therefore relates to a biocompatible non-porous matrix based on chitosan and an acid, in particular a hydroxy carboxylic acid, which may be for example in the form of a sheet or of a three-dimensional article, e.g. of a hollow article or of a roll. The non-porous matrix can be obtained by:
providing an aqueous solution of a chitosan and an acid, in particular a hydroxy carboxylic acid, which is present in excess,
drying the solution without freezing and
removing excess acids before or/and after drying, preferably by neutralization.

The non-porous matrix can be used as carrier for a porous three-dimensional matrix. It is thus possible to provide biocompatible matrix systems which comprise at least one biocompatible non-porous matrix as described previously, and at least one biocompatible porous matrix. The structure of the biocompatible porous matrix is preferably based on chitosan and an acid, in particular a hydroxy carboxylic acid. However, it is also possible to use other porous biocompatible matrices.

A biocompatible porous matrix as disclosed in German application 199 48 120.2 is particularly preferred and is obtainable by:
providing an aqueous solution of a chitosan and of an acid, in particular a hydroxy carboxylic acid, which is present in excess,
freezing and drying the solution, in particular by sublimation under reduced pressure, and
removing excess acid before or/and after the freezing, in particular by neutralization with a suitable base, e.g. NaOH.

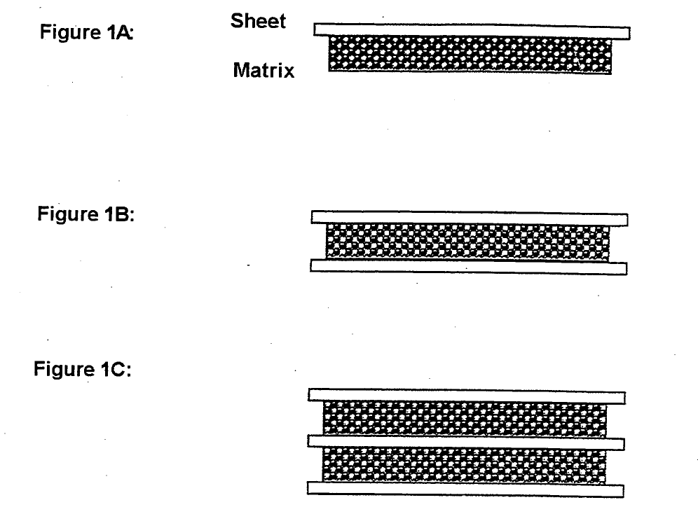

In matrix system of the invention it is possible for non-porous matrices and porous matrices each to be disposed alternately in layers. Examples of such multilayer systems are depicted in FIGS. 1A, 1B and 1C. As an alternative, a non-porous matrix can also be disposed between two porous matrices.

The non-porous matrix of the invention or the matrix system based thereon can be used for the in vitro culturing of cells. In this case, the matrix system may comprise additional factors for cell growth, e.g. cytokines.

The matrix or the matrix system can be employed for example for culturing cartilage tissue, for reconstructing bone tissue, as filling material for bioreactors for producing cells, proteins or viruses, as microcarrier of filling material for bioreactors, for generating capillaries and blood vessels, for generating optionally multilayer skin systems, for culturing blood stem cells, for regenerating nerve tissues and for generating artificial organs.

A particularly preferred application of the multilayer matrix system is the production of a base material for generating a multilayer artificial skin system. In this case, the matrix system may be colonized by keratinocytes and, where appropriate, additionally by fibroblasts. A further possibility is to generate a vascularized skin system, in which case tubes are drawn into the porous layers of the matrix system which, after colonization with epithelial cells, contribute to the vascularization of the artificial skin.

A further particularly preferred application of the multilayer matrix system is the generation of an artificial heart valve, in which case a non-porous structure is incorporated between two porous structures, to increase the mechanical stability, and is then used for culturing muscle cells.

A further possibility is to employ the non-porous matrix and the matrix system based thereon also as implant without previous cell colonization, e.g. for cartilage and bone defects, as substitute for microcapillaries or as surgical filling material, e.g. for reconstructive surgery or cosmetic surgery.

A further aspect of the present invention relates to a biocompatible matrix based on chitosan and an acid, in particular a hydroxy carboxylic acid with anisotropic structures, for example fibers or/and chambers in parallel alignment. In this embodiment, the matrix is preferably porous. The anisotropic matrix can be obtained by:
providing an aqueous solution of a chitosan and of an acid, in particular a hydroxy carboxylic acid, which is present in excess,
anisotropic freezing and drying of the solution, in particular by sublimation under reduced pressure, and
removing excess acid before or/and after freezing.

The anisotropic freezing preferably comprises a freezing with use of structured cooling elements, e.g. tubes in direct or indirect contact with the matrix during the freezing process. The cooling elements may be elongate in order to obtain for example fibers or chambers in parallel alignment in the matrix. However, it is also possible to use curved structures, e.g. simulations of the organ to be shaped, as cooling elements.

The anisotropic porous matrix can be employed in a biocompatible matrix system together with another matrix, for example with a biocompatible non-porous matrix. The anisotropic matrix or the matrix system based thereon can be employed for the in vitro culturing of cells or as implant without previous cell colonization in accordance with the aforementioned applications.

Yet a further aspect of the invention is the use of a biocompatible matrix based on chitosan and an acid, in particular a hydroxy carboxylic acid, as described in DE 199 48 120.2, for culturing cartilage tissue, for reconstructing bone tissue, as filling material for bioreactors for producing cells, proteins or viruses, as microcarrier of filling material for bioreactors, for generating capillaries and blood vessels, for generating optionally multilayer skin systems, for culturing blood stem cells, for regenerating nerve tissues, for generating artificial organs.

It has surprisingly been found that cells can be cultured in a density of 10⁶ or more cells per cm² of matrix. This cell density is an increase of more than ten-fold compared with culturing in a culture dish.

The matrices of the invention based on chitosan and acids are essentially produced by the method indicated in German application 199 48 120.2 unless stated otherwise. Preferably, first an aqueous solution of a partially deacetylated chitosan and of an acid which is present in excess is prepared. Excess means in this connection that the pH of the aqueous solution is in the acidic range, preferably below pH≦4. The free amino groups of the chitosan are at least partially protonated thereby, thus increasing the solubility in water. The amount of acid is not critical. It needs merely to be chosen so that the chitosan dissolves. Excessive addition of acid is avoided as far as possible because excess acid must be removed again, and working up is impeded with large amounts of acid thereby. Favorable amounts of acid result in a 0.05 to 1 N, preferably 0.1 to 0.5 N, in particular 0.1 to 0.3 N, solution. The amount of chitosan is preferably chosen to result in a 0.01 to 0.5 M, preferably 0.1 to 0.3 M, solution. The structure of the matrix, especially the pore size thereof, can be influenced via concentration of the chitosan solution. It is possible in this way to adjust the pore size of the matrix to the particular cell type of which the matrix is to be colonized.

Because chitosan is produced from natural sources it has no uniform molecular weight. The molecular weight may be between 20 kDa to more than 1000 kDa depending on the source and method of processing.

The chitosan for producing the three-dimensional matrix is not subject to any restrictions in relation to its molecular weight. The aqueous chitosan solution is produced by using an acid which is an inorganic acid or, preferably, an organic acid, particularly preferably an alkyl or aryl hydroxy carboxylic acid. Hydroxy carboxylic acids having 2 to 12 carbon atoms are particularly suitable, it being possible for one or more hydroxyl groups and one or more carboxyl groups to be present in the molecule. Specific examples are glycolic acid, lactic acid, malic acid, tartaric acid, citric acid and mandelic acid. Lactic acid is particularly preferred.

In producing a porous matrix, the solution of chitosan and acid is initially at least partially neutralized by adding base and then frozen or directly frozen without previous neutralization. Neutralization before freezing is preferred. The pH after the neutralization is generally 5.0 to 7.5 , preferably from 5.5 to 7.0 and in particular from 6.0 to 7.0.

After the freezing, the water is removed by sublimation under reduced pressure, for example in the pressure range from 0.001 to 3 hPa.

To produce a non-porous matrix, the solution is not subjected to freezing and sublimation, but is dried without freezing at optionally elevated temperature or/and reduced pressure, and is preferably neutralized after drying. The resulting non-porous matrix has a high load-bearing capacity and extensibility in the moist state.

The large number of amino and hydroxyl groups makes the matrix modifiable as desired. In a preferred embodiment of the three-dimensional matrix, ligands are covalently or noncovalently bound to the chitosan matrix, preferably to the free amino groups of chitosan. Ligands which can be used are, for example, growth promoters, proteins, hormones, heparin, heparan sulfates, chondroit sulfates, dextran sulfates or a mixture of these substances. The ligands preferably serve to control and improve cell proliferation.

The ligands used in the matrix in a preferred embodiment of the invention are nucleic acids, e.g. RNA or DNA. The nucleic acids can be immobilized by chemical coupling to the amino or/and hydroxyl groups present in the chitosan. It is possible with a nucleic acid-loaded matrix to achieve locally restricted transient expression of heterologous genes in the body. This is because when a matrix coupled in this way is implanted in the body and colonized by endogenous cells which dissolve the matrix, the cells also take up the nucleic acids immobilized thereon and are able to express the latter.

Cell growth on the matrix is further improved if the matrix is cultured with autologous fibrin.

The three-dimensional matrix of the invention can be used as solid phase in a culture reactor (Cell Factory) . The matrix shows a very high resistance in the culture medium. It has also emerged that the matrix promotes cell growth.

The matrix is further suitable for use as cell implant, in particular for cartilage-forming cells. No genetically modified cells must be used in this case.

The cells are preferably taken from the patient by biopsy and cultured on the cell matrix, and the cell implant is then implanted into the patient. Transplant rejection reactions are substantially precluded owing to the colonization of the three-dimensional matrix with endogenous stem cells (bone substitute) which, stimulated by the respective growth factors of the surrounding tissue, differentiate only at the site of the transplant, or with cartilage cells for renewed formation of hyaline cartilage.

The three-dimensional matrix can be colonized both by human and by animal cells (for example from horse, dog or shark). Shark cells are particularly suitable because they induce negligible immunological response in the recipient. Shark cells are already used as organ replacement, e.g. for the lenses of eyes. Examples of cells with which the matrices or matrix systems of the invention can be colonized are chondrocytes, osteocytes, keratinocytes, hepatocytes, bone marrow stem cells or neuronal cells.

The matrices or matrix systems as described previously can be employed in the human medical and veterinary sectors. Further areas of application are the use as disposable article as in vitro test system for investigating active pharmaceutical ingredients. For this purpose, for example, blood stem cells or hepatocytes can be cultured on the matrix. This system can be used to investigate the activity of test substances from a chemical or/and biological substance library, where appropriate in a high-throughput method.

The matrix and the matrix system are sterilized before use in the cell culture, in order to guarantee freedom from germs. The sterilization can take place by thermal treatment, e.g. by autoclaving, steam treatment etc. or/and by irradiation, e.g. gamma-ray treatment. The sterilization is preferably carried out in a physiologically tolerated buffered solution, e.g. in PBS, in order to ensure thorough wetting of the matrix with liquid and the absence of larger air inclusions.

When the cells are cultured, the matrix is degraded within a period of about 5-8 weeks. The degradation time can be adjusted via the degree of the deacetylation of the chitosan and the concentration of the material.

The invention is further to be explained by the following examples.

### EXAMPLE 1

### Production of a Non-Porous Sheet

A mixture of chitosan and lactic acid is prepared by the method described in Example 3 of DE 199 48 120.2. The solution is poured into a Petri dish and dried at 50° C. and, after a glass-clear film has resulted, neutralized to a pH of 7 with 1 M sodium hydroxide solution. The resulting sheet has a high load-bearing capacity and extensibility in the moist state.

### EXAMPLE 2

### Growth of Hep-G2 Cells in the Matrix

Two defined initial amounts, 1×10⁵ and 1×10⁶, of Hep-G2 hepatocytes were injected into a piece, 1.5 cm² in size, of porous matrix (produced as in Example 3 of DE 199 48 120.2), and cell growth was observed at four points in time for a maximum of 33 days. A continuous cell growth was observable in this case.

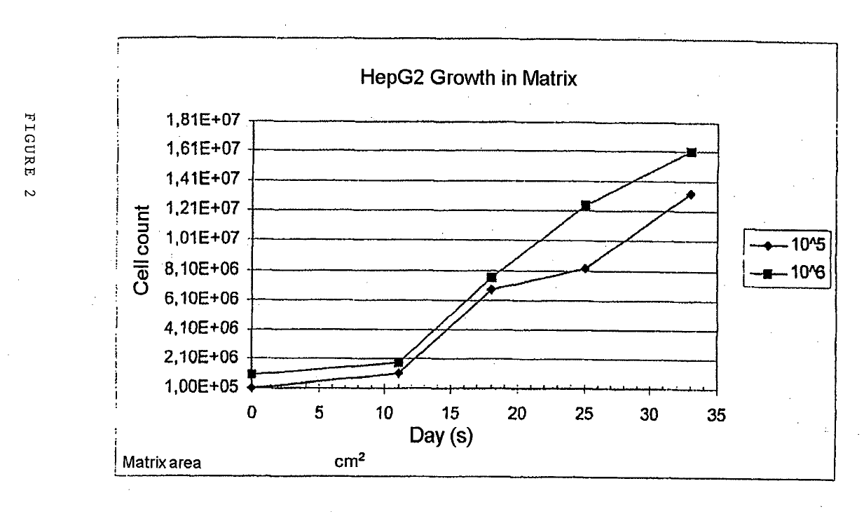

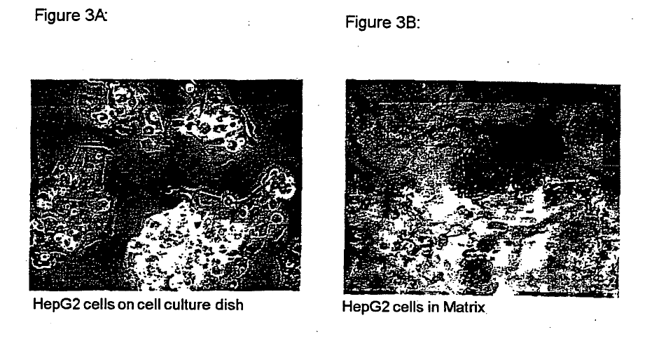

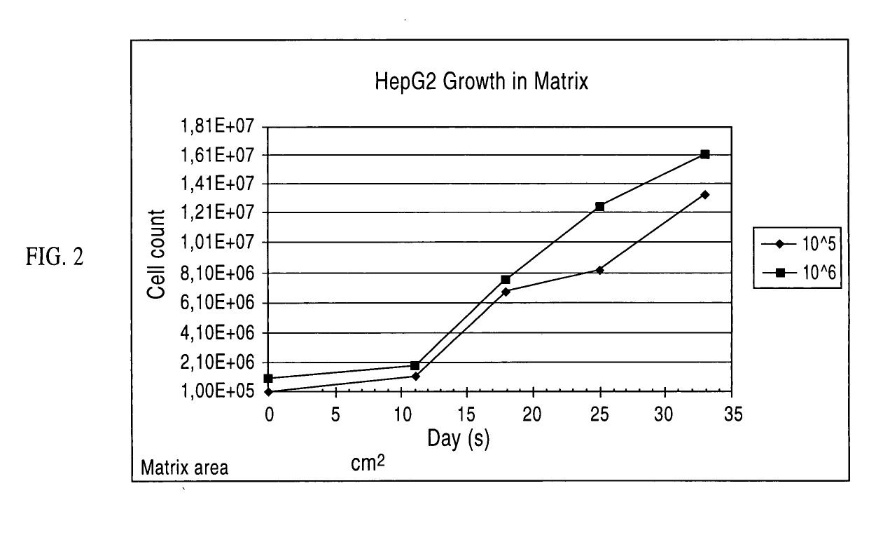

The maximum cell count per matrix after 33 days was 1.6×10⁷ cells (FIG. 2). This means the cell count was able to increase further by one power of ten on the small basic area of 1.5 cm². The cell density of a confluent, conventional culture dish with a basic area of 80 cm² is stated by the Deutsche Sammlung von Mikroorganismen und Zellkulturen (DSMZ) to be 2.5-3.0×10⁷ Hep-G2. This amount is, when apportioned to the basic area of the matrix, about 25 times less than the cell count determinable in the matrix after 33 days.

### EXAMPLE 3

### Effect of the Matrix on Cell Proliferation

The intention of this experiment was to show whether substances present in the matrix have an unfavorable influence on cell growth. It was intended in this case to assess not the growth of the cells on the matrix, but only the influence of potential soluble substances possibly released into the medium. For this purpose, a piece, 1.5 cm² in size, of a matrix (produced as in Example 3 of DE 199 48 120.2) was preincubated in 3 ml of cell culture medium at 37° C. and 5% CO₂ for 6 days. The medium was then analyzed with control media, which had likewise been preincubated, in a standard proliferation assay (XTT). In this assay, a tetrazolium salt is converted by metabolically active cells into a colored formazan salt which can subsequently be detected by photometry. No influence on cell growth was observable in this case. Hep-G2 was used as cell line, and 5% DMSO was added to the medium as positive control. The assay was repeated three times and gave the same result in all three cases.

### EXAMPLE 4

### Growth of other Cell Lines in the Matrix and Cell Morphology

Besides Hep-G2, two other cell lines were seeded on the matrix in order to observe whether they grow in the matrix. Both Hela and the CHO-K1 cell line is able to grow in the matrix.

An altered morphology compared with cells growing in normal culture dishes is observable with all three cell lines. The cells are distinctly rounded and also grow in the third dimension and thus show more resemblance to cells in natural three-dimensional tissues. As example, FIG. 3 shows two pictures of the hepatocyte line Hep-G2 with FIG. 3A showing the cells after culturing from a cell culture dish and FIG. 3B showing the cells after culturing in a matrix.

1-50. (canceled)

51. A biocompatible non-porous matrix based on chitosan and an acid, wherein said matrix is produced by:
providing an aqueous solution comprising a chitosan and an acid, wherein said acid is present in excess;
drying the solution without freezing; and
removing excess acid before or/and after the drying.

52. The non-porous biocompatible matrix of claim 51, wherein the acid is a hydroxy carboxylic acid.

53. The non-porous biocompatible matrix of claim 51, wherein the matrix is in the form of a sheet, a hollow article, or a roll.

54. The non-porous biocompatible matrix of claim 52, wherein the hydroxy carboxylic acid is a member selected from the group consisting of glycolic acid, lactic acid, malic acid, tartaric acid, citric acid and mandelic acid.

55. The non-porous biocompatible matrix of claim 54, wherein the hydroxy carboxylic acid is lactic acid

56. A biocompatible matrix system comprising at least one biocompatible non-porous matrix as claimed in claim 51 and at least one biocompatible porous matrix.

57. The biocompatible matrix system of claim 56, wherein the at least one biocompatible porous matrix has a structure based on chitosan and an acid.

58. The biocompatible matrix system of claim 57, wherein the acid of the porous matrix is a hydroxy carboxylic acid.

59. The biocompatible matrix system of claim 57, wherein the porous matrix is produced by:
providing an aqueous solution comprising a chitosan and an acid, wherein said acid is present in excess;
freezing and drying the solution; and
removing excess acid before or/and after the freezing.

60. The biocompatible matrix system of claim 59, wherein the acid is a hydroxy carboxylic acid.

61. The biocompatible matrix system of claim 59, wherein the drying is achieved by sublimation under reduced pressure.

62. The biocompatible matrix system of claim 56, wherein the at least one non-porous matrix and the at least one porous matrix are disposed alternatively in layers.

63. A method for culturing cells in vitro, said method comprising:
obtaining cells; and
culturing the cells on the non-porous matrix of claim 51.

64. The method of claim 63, wherein the matrix system comprises a ligand.

65. The method of claim 64, wherein the ligand is a factor for cell growth.

66. The method of claim 63, wherein the cells are obtained from cartilage, bone, blood vessel tissue, skin, or nerve tissue.

67. The method of claim 63, wherein the matrix is a bioreactor filling material for producing cells, proteins, or viruses.

68. The method of claim 63, wherein the matrix is a microcarrier of filling material for a bioreactor.

69. The method of claim 66, wherein the blood vessel tissue provides for capillary generation.

70. The method of claim 63, wherein the cells are blood stem cells.

71. The method of claim 63, wherein the matrix provides for artificial organ generation.

72. The method of claim 63, wherein the matrix provides for skin system generation.

73. The method of claim 72, wherein the matrix is multilayered.

74. A method for repairing a cartilage or bone defect, said method comprising implanting the non porous matrix of claim 51 in the area of a bone or cartilage defect in a patient, wherein the matrix is without previous cell colonization.

75. A method for replacing a microcapillary in a patient, said method comprising introducing the non porous matrix of claim 51, in the form of a microcapillary, in a patient, wherein the matrix is without previous cell colonization.

76. A method for providing a filler material during surgery comprising implanting the non porous matrix of claim 51 in a patient in need of such filler, wherein the matrix is without previous cell colonization.

77. A biocompatible matrix having anisotropic structures, said matrix comprising chitosan and an acid.

78. The anisotropic biocompatible matrix of claim 77, wherein the acid is a hydroxy carboxylic acid.

79. The anisotropic biocompatible matrix of claim 77, wherein said matrix comprises fibers or chambers in parallel alignment.

80. The anisotropic biocompatible matrix of claim 77, wherein said matrix is porous.

81. The anisotropic biocompatible matrix of claim 77, wherein said matrix is produced by:
providing an aqueous solution comprising a chitosan and an acid, wherein the acid is present in excess,
providing anisotropic freezing and drying of the solution,
removing excess acid before or/and after the freezing.

82. The anisotropic biocompatible matrix of claim 81, wherein the acid is a hydroxy carboxylic acid.

83. The anisotropic biocompatible matrix of claim 81, wherein the drying is achieved by sublimation under reduced pressure.

84. A biocompatible matrix system comprising at least one biocompatible anisotropic porous matrix as claimed in claim 77 and at least one biocompatible non-porous matrix.

85. A method for culturing cells in vitro, said method comprising:
obtaining cells; and
culturing the cells on the anisotropic matrix of claim 77.

86. A method for repairing a cartilage or bone defect, said method comprising implanting the matrix of claim 77 in the area of a bone or cartilage defect in a patient, wherein the matrix is without previous cell colonization.

87. A method for replacing a microcapillary in a patient, said method comprising introducing the matrix of claim 77, in the form of a microcapillary, in a patient, wherein the matrix is without previous cell colonization.

88. A method for providing a filler material during surgery comprising implanting the matrix of claim 77 in a patient in need of such filler, wherein the matrix is without previous cell colonization.

89. A biocompatible matrix based on chitosan and an acid, wherein said matrix comprises nucleic acids in chemically coupled-on form.

90. The biocompatible matrix of claim 89, wherein the acid is a hydroxy carboxylic acid.

91. A method for culturing cells in vitro, said method comprising:
obtaining cells; and
culturing the cells on a biocompatible matrix based on chitosan and an acid.

92. The method of claim 91, wherein the acid is a hydroxy carboxylic acid.

93. The method of claim 91, wherein the cells are obtained from cartilage, bone, blood vessel tissue, skin, or nerve tissue.

94. The method of claim 91, wherein the matrix is a bioreactor filling material for producing cells, proteins, or viruses.

95. The method of claim 91, wherein the matrix is a microcarrier of filling material for a bioreactor.

96. The method of claim 93, wherein the blood vessel tissue provides for capillary generation.

97. The method of claim 91, wherein the cells are blood stem cells.

98. The method of claim 91, wherein the matrix provides for artificial organ generation.

99. The method of claim 91, wherein the matrix provides for skin system generation.

100. The method of claim 99, wherein the matrix is multilayered.

101. The method of claim 91, wherein the matrix is produced by:
providing an aqueous solution comprising a chitosan and an acid, wherein said acid is present in excess;
freezing and drying the solution; and
removing excess acid before or/and after the freezing.

102. The method of claim 101, wherein the acid is a hydroxy carboxylic acid.

103. The method of claim 101, wherein the drying is achieved by sublimation under reduced pressure.

104. The method of claim 91, wherein the matrix is sterilized.

105. The method of claim 91, wherein the cells are cultured in a density of 10⁶ or more cells per cm² on or in the matrix.

106. A method for culturing cells in vitro, said method comprising:
obtaining cells; and
culturing the cells on the matrix system of claim 56.

107. A method for repairing a cartilage or bone defect, said method comprising implanting the matrix system of claim 56 in the area of a bone or cartilage defect in a patient, wherein the matrix system is without previous cell colonization.

108. A method for replacing a microcapillary in a patient, said method comprising introducing the matrix system of claim 56, in the form of a microcapillary, in a patient, wherein the matrix system is without previous cell colonization.

109. A method for providing a filler material during. surgery comprising implanting the matrix system of claim 56 in a patient in need of such filler, wherein the matrix system is without previous cell colonization.

110. A method for culturing cells in vitro, said method comprising:
obtaining cells; and
culturing the cells on the matrix system of claim 84.

111. A method for repairing a cartilage or bone defect, said method comprising implanting the non porous matrix of claim 84 in the area of a bone or cartilage defect in a patient, wherein the matrix system is without previous cell colonization.

112. A method for replacing a microcapillary in a patient, said method comprising introducing the matrix system of claim 84, in the form of a microcapillary, in a patient, wherein the matrix system is without previous cell colonization.

113. A method for providing a filler material during surgery comprising implanting the matrix system of claim 84 in a patient in need of such filler, wherein the matrix system is without previous cell colonization.
